(19) United States
(12) Patent Application Publication
(10) Pub. No.: US 2026/0273199 A1
(43) Pub. Date: Sep. 17, 2026

(54) SYSTEM FOR AUTOMATIC DETECTION, MEASUREMENT, AND ELIMINATION OF AIR FROM A FLUID PATHWAY

(71) Applicant: Pneuma Systems Corporation, Portsmouth, NH (US)

(72) Inventor: Jeffrey A. Carlisle, Portsmouth, NH (US)

(21) Appl. No.: 19/565,109

(22) Filed: Mar. 12, 2026

Related U.S. Application Data
(60) Provisional application No. 63/770,488, filed on Mar. 12, 2025.

(51) Int. Cl. A61M 5/36; A61M 5/40

(52) U.S. Cl. CPC A61M 5/365; A61M 5/40

(57) ABSTRACT

A system for detecting, measuring, and removing gas from a medical fluid pathway includes a fluid delivery path that conveys therapeutic liquid from a source toward a patient and an Active Air Management (AAM) subassembly. The AAM subassembly includes a fluid chamber that receives the therapeutic liquid and entrained gas, and a pneumatic chamber separated from the fluid chamber by a gas-selective membrane that permits gas transfer while retaining liquid. A pressure controller is fluidly coupled to the pneumatic chamber and configured to impose a controlled negative pressure differential to extract gas across the membrane. A valve selectively isolates the pneumatic chamber to create a sealed measurement volume, and a pressure sensor measures pressure changes within the sealed volume. A controller determines a quantity of gas based on the measured pressure change and, when the change meets or exceeds a threshold, commands the pressure controller to re-establish or adjust the negative pressure differential to continue gas removal.

### CROSS REFERENCE TO RELATED APPLICATIONS

This application claims the benefit of U.S. Provisional Patent Application No. 63/770,488, filed Mar. 12, 2025, the contents of which is incorporated herein by reference in its entirety as if fully set forth.

### FIELD OF THE INVENTION

Illustrative embodiments of the invention generally relate to fluid delivery systems and, more particularly, illustrative embodiments relate to devices and methods for safely and effectively infusing fluids and medications to patients intravenously.

### BACKGROUND OF THE INVENTION

In intravenous infusion therapy, fluids must be delivered to the patient without any significant air bubbles. Air volume greater than 1,000 μL over 15 minutes can cause harm to an adult. Neonates and/or patients with certain cardiac defects are especially sensitive to inline bubbles, and can be harmed at even smaller air volumes.

### SUMMARY OF VARIOUS EMBODIMENTS

In accordance with one embodiment, a system for detecting, measuring, and removing gas from a medical fluid pathway includes a fluid delivery path that conveys therapeutic liquid from a source toward a patient and an Active Air Management (AAM) subassembly. The AAM subassembly includes a fluid chamber that receives the therapeutic liquid and entrained gas, and a pneumatic chamber separated from the fluid chamber by a gas-selective membrane that permits gas transfer while retaining liquid. A pressure controller is fluidly coupled to the pneumatic chamber and configured to impose a controlled negative pressure differential to extract gas across the membrane. A valve selectively isolates the pneumatic chamber to create a sealed measurement volume, and a pressure sensor measures pressure changes within the sealed volume. A controller determines a quantity of gas based on the measured pressure change and, when the change meets or exceeds a threshold, commands the pressure controller to re-establish or adjust the negative pressure differential to continue gas removal.

In some embodiments, the pressure controller is configured to impose a negative gauge pressure within the pneumatic chamber relative to the fluid chamber to draw gas across the gas-selective membrane.

In some embodiments, the controller is configured to isolate the pneumatic chamber using the valve while the pneumatic chamber is at the negative gauge pressure, and to calculate the gas quantity based on a rise in pressure within the sealed measurement volume.

In some embodiments, the controller is configured to control the pressure controller to re-establish the negative gauge pressure when the measured pressure within the pneumatic chamber deviates from a target pressure by more than a predetermined percentage.

In some embodiments, the pressure controller is configured to impose a positive gauge pressure within the pneumatic chamber relative to atmospheric pressure.

In some embodiments, the positive gauge pressure establishes a pressure differential across the gas-selective membrane that promotes transfer of gas from the fluid chamber into the pneumatic chamber while substantially preventing passage of liquid.

In some embodiments, the controller is configured to calculate the gas quantity based on a change in the positive gauge pressure within the pneumatic chamber when the pneumatic chamber is isolated by the valve.

In some embodiments, the pressure controller comprises a piezoelectrically actuated microblower configured to generate oscillating air pressure that is rectified to produce the controlled pressure differential.

In some embodiments, the controller is configured to calculate the gas quantity using a known reference volume of the pneumatic chamber and a change in measured pressure in accordance with a gas law relationship.

In some embodiments, the system further comprises a communication interface configured to transmit at least one of calculated gas quantity, pressure data, or system status information to an infusion control device.

In accordance with one embodiment, a system for detecting, measuring, and removing gas from a medical fluid pathway includes a fluid delivery path configured to convey therapeutic liquid from a fluid source toward a patient. The system further includes a fluid chamber forming at least a portion of the fluid delivery path and configured to receive therapeutic liquid and entrained gas. A gas-selective membrane is positioned adjacent to the fluid chamber and permits gas to pass therethrough while retaining liquid. A pressurized chamber containing a pressurizing medium is arranged to apply positive pressure to at least a portion of the fluid delivery path and to a controllable fluid restrictor, thereby establishing a pressure differential across the gas-selective membrane that promotes transfer of gas from the fluid chamber through the membrane. A pressure controller regulates the positive pressure applied by the pressurized chamber, and a fluid flow resistance control device creates hydrodynamic backpressure. The system further includes a pressure sensor configured to measure pressure associated with the pressurized chamber or the fluid chamber, and a controller configured to calculate a gas quantity based on a change in the measured pressure.

In some embodiments, the pressurized chamber surrounds at least a portion of the fluid chamber and transmits pressure to the therapeutic liquid through a compliant wall or separating membrane.

In some embodiments, the gas-selective membrane is positioned adjacent to a narrowed fluid passage configured to mechanically constrain air bubbles and increase contact between the air bubbles and the gas-selective membrane.

In some embodiments, the system further comprises a variable flow restrictor positioned downstream of the fluid chamber and configured to selectively adjust flow resistance in the fluid delivery path.

In some embodiments, the controller is configured to increase downstream flow resistance to temporarily reduce liquid flow while gas is being transferred across the gas-selective membrane.

In some embodiments, the system further comprises a one-way check valve positioned downstream of the gas-selective membrane and configured to prevent retrograde gas flow into the fluid chamber.

In some embodiments, the pressure controller comprises a pump configured to generate positive gauge pressure within the pressurized chamber.

In some embodiments, the controller is configured to calculate the gas quantity using a known reference volume and a pressure change in accordance with a gas law relationship.

In some embodiments, the controller is configured to communicate calculated gas quantity data to an infusion control device.

In accordance with one embodiment, a method for detecting, measuring, and removing gas from a medical fluid pathway includes conveying a therapeutic liquid from a fluid source toward a patient through a fluid delivery path and directing the therapeutic liquid and any entrained gas into a fluid chamber in fluid communication with the fluid delivery path. Gas is drawn from the fluid chamber into a pneumatic chamber through an integral gas-selective membrane that allows gas to pass while retaining liquid in the fluid chamber by imposing and maintaining a controlled negative pressure differential between the pneumatic chamber and the fluid chamber using a pressure controller. The pneumatic chamber is selectively isolated from the pressure controller using a valve to create a sealed measurement volume, pressure changes within the sealed measurement volume are measured using a pressure sensor, and a controller calculates a gas quantity corresponding to the measured pressure change. In response to the pressure change meeting or exceeding a threshold, the controller causes the pressure controller to re-establish or adjust the controlled pressure differential in the pneumatic chamber.

In some embodiments, the method includes imposing a negative gauge pressure within the pneumatic chamber relative to the fluid chamber to draw gas across the gas-selective membrane.

In some embodiments, the method includes isolating the pneumatic chamber using the valve while the pneumatic chamber is at the negative gauge pressure and calculating a gas quantity based on a rise in pressure within the sealed measurement volume.

In some embodiments, the method includes controlling the pressure controller to re-establish the negative gauge pressure when the measured pressure within the pneumatic chamber deviates from a target pressure by more than a predetermined percentage.

In some embodiments, the method includes imposing a positive gauge pressure within the pneumatic chamber relative to atmospheric pressure using the pressure controller.

In accordance with another embodiment, a system removes gas from a medical fluid delivery pathway. The system may include a fluid delivery path configured to convey a medical fluid from a fluid source toward a patient. An air removal assembly may be positioned along the fluid delivery path and may include a fluid chamber configured to receive the medical fluid together with any entrained gas bubbles. The air removal assembly may further include a pneumatic chamber that is separated from the fluid chamber by a gas-selective membrane. The gas-selective membrane permits passage of gas from the fluid chamber into the pneumatic chamber while inhibiting the passage of liquid. A flow path region may be positioned adjacent to the gas-selective membrane and configured to direct the flowing medical fluid along the surface of the membrane so that gas bubbles entrained in the fluid are compelled to contact the membrane.

The system may further include a pressure control system configured to actively establish and maintain a pressure differential between the fluid chamber and the pneumatic chamber such that the pressure on the fluid side of the gas-selective membrane exceeds the pressure on the gas side of the membrane. This pressure relationship drives gas from the medical fluid across the membrane and into the pneumatic chamber. Gas that passes into the pneumatic chamber may then be removed from the system through a gas evacuation pathway coupled to the pneumatic chamber, allowing the gas to exit the device while the degassed medical fluid continues along the fluid delivery path toward the patient.

The pressure control system may be configured to generate a sub-atmospheric pressure within the pneumatic chamber. By establishing a vacuum on the gas side of the gas-selective membrane, the system creates a pressure differential across the membrane such that the pressure within the fluid chamber exceeds the pressure within the pneumatic chamber. This differential pressure drives gas from entrained bubbles across the membrane and into the pneumatic chamber. In other embodiments, the pressure control system may instead apply positive pressure to the fluid chamber or to a fluid region upstream of the gas-selective membrane. Maintaining the fluid chamber at a pressure greater than the pressure on the gas side of the membrane similarly produces the required pressure differential, causing gas contacting the membrane surface to pass through the membrane while the liquid phase remains within the fluid pathway.

In further embodiments, the system may include sensing and control components configured to detect and quantify gas removal. For example, a pressure sensor may be configured to detect pressure changes within the pneumatic chamber, and a controller may be configured to determine a gas volume based on those pressure changes using known chamber characteristics and gas relationships. In some implementations, a valve may be provided to selectively isolate the pneumatic chamber, thereby forming a sealed reference volume that enhances measurement sensitivity when gas passes through the membrane into the chamber. In additional embodiments, gas transfer may be detected through monitoring of pneumatic actuator performance. For instance, the pressure control system may include a pneumatic actuator such as a microblower, pump, or similar device, and a controller may be configured to detect gas transfer events by identifying variations in electrical power consumption of the actuator that correspond to airflow or pneumatic work associated with gas passing across the membrane.

In various embodiments, the pressure differential across the gas-selective membrane may be established in different ways. For example, the pressure control system may reduce the pressure in the pneumatic chamber relative to the fluid chamber, thereby creating a vacuum condition on the gas side of the membrane that draws gas across the membrane. In other embodiments, the pressure control system may increase the pressure within the fluid chamber relative to the pneumatic chamber to establish the required pressure differential. In some implementations, the pressure control system may include a pneumatic actuator capable of selectively generating both positive pressure and vacuum conditions as required during operation. Such an actuator may comprise, for example, a microblower or other compact pneumatic device capable of producing controlled bidirectional pressure differentials.

In certain embodiments, the flow path region adjacent the gas-selective membrane may include a constricted channel that guides the fluid along the membrane surface. The constricted channel may have a cross-sectional dimension selected to deform gas bubbles entrained in the fluid, thereby increasing the surface area of contact between the bubbles and the membrane. In other embodiments, the flow path region may include a tortuous or otherwise shaped pathway designed to increase the probability and duration of bubble contact with the membrane. In some implementations, the gas-selective membrane itself may form at least a portion of the wall defining the flow path region so that fluid flowing through the channel travels directly along the membrane surface.

The gas-selective membrane may take various forms depending on the desired operating conditions. In some embodiments the membrane may comprise a hydrophobic membrane configured to allow gas passage while inhibiting liquid penetration. In other embodiments, the membrane may comprise an omniphobic membrane configured to resist wetting by multiple liquid types, including fluids with varying surface tensions or chemical compositions. In certain implementations, the gas-selective membrane may include multiple membrane segments arranged to increase the available surface area for gas transfer. As described previously, certain embodiments may omit a hydrophilic liquid-transfer membrane entirely, relying instead on the controlled pressure differential and fluid path geometry to achieve gas separation.

In various embodiments, gas that passes through the gas-selective membrane may exit the system through a gas evacuation pathway. The evacuation pathway may include a one-way check valve configured to prevent external air from entering the pneumatic chamber. In some implementations, gas may simply vent to atmosphere through this pathway, while in other implementations the gas may be directed into a sealed gas collection chamber or other containment volume.

In certain embodiments, the system may further include sensing and control features for detecting and quantifying gas removal events. For example, a pressure sensor may detect pressure changes within the pneumatic chamber, and a controller may determine a corresponding gas volume based on those pressure changes. The controller may perform this calculation using gas-law relationships based on the measured pressure change and the known volume of the chamber. In some implementations, a valve may selectively isolate the pneumatic chamber so that it forms a sealed reference volume for measurement of gas transfer across the membrane. In addition to pressure-based sensing, certain embodiments may detect gas transfer by monitoring the power consumption of the pneumatic actuator. Because pneumatic work increases when gas flows through the system, transient increases in actuator power consumption may correspond to gas bubbles passing across the membrane.

In various embodiments, a controllable flow restrictor may be positioned downstream of the gas-selective membrane. A controller may adjust this restrictor to regulate the infusion rate and maintain the desired pressure differential across the membrane during operation. Because gas removal is driven by pressure differential rather than buoyancy, the system may operate effectively regardless of the orientation of the fluid delivery path relative to gravity. The device may therefore function in vertical, horizontal, inverted, or mobile orientations. In certain implementations, the geometry of the flow path region may be configured to compel bubbles above a selected volume threshold to contact the gas-selective membrane, ensuring reliable removal of clinically significant bubbles. For example, the selected threshold volume may be less than approximately 50 microliters.

### BRIEF DESCRIPTION OF THE DRAWINGS

Those skilled in the art should more fully appreciate advantages of various embodiments from the following “Description of Illustrative Embodiments,” discussed with reference to the drawings summarized immediately below.

FIG. 1 schematically shows an example system in accordance with various embodiments.

FIG. 2 schematically shows an example mechanical construction of a filter subassembly in accordance with various embodiments.

FIG. 3 schematically shows an example pneumatic control subsystem in accordance with various embodiments.

FIG. 4 shows an example process of automatic detection, measurement, and elimination of air from a fluid pathway in accordance with various embodiments.

FIG. 5 shows an example system in accordance with various embodiments.

FIG. 6 schematically shows an example active gas monitoring, detecting, and measuring in accordance with various embodiments.

FIG. 7 schematically shows an example passive gas monitoring, detection, and measurement in accordance with various embodiments.

FIG. 8 schematically shows example details of a controller of FIG. 1 configured in accordance with various embodiments.

### DESCRIPTION OF ILLUSTRATIVE EMBODIMENTS

In illustrative embodiments air bubbles are detected, measured, and removed during the delivery of critical liquids, such as through intravenous therapy, to ensure patient safety. Various embodiments use an actively generated pneumatic pressure differential across a gas-selective membrane, such as a hydrophobic membrane, together with a fluid-path geometry to cause entrained air bubbles to come into contact with the membrane. The gas is thus selectively removed from the flowing liquid stream without permitting liquid passage through the membrane. The pneumatic pressure differential may be created by applying a vacuum on one side of the membrane. Alternatively the pressure differential may be created by maintaining positive pressure on the fluid side of the membrane relative to the gas side. In either case, active pneumatic control establishes the driving force for gas transfer, while the narrow and/or tortuous fluid path ensures that bubbles physically engage the membrane surface, which is necessary for removal. Details of illustrative embodiments are discussed below.

Gas (e.g., air bubbles) can enter a fluid pathway system in several ways. It may be present in large volumes (several milliliters), as smaller bubbles measuring dozens of microliters, or as highly dispersed “champagne bubbles.” Gas may also be introduced when fluid sources are connected to a delivery system.

Within fluid pathways—such as flexible tubing or connectors like stopcocks—gas often becomes intermingled with liquid. In these confined spaces, surface tension governs the gas-liquid interface. As a result, gas does not necessarily rise to the highest point or follow a predictable path through the system.

Over time, changes in temperature and pressure can also cause dissolved gases to come out of a solution, a process known as outgassing. Some solutions are more susceptible to this effect than others, and certain fluids may generate bubbles under normal conditions of use.

During intravenous drug delivery, clinicians often aim to administer the full contents of a bag, bottle, or vial. However, completing an infusion without introducing air requires significant attention and time. Additionally, many infusion containers include air spaces—typically at the top of a bag, bottle, or drip chamber. If these containers are jostled or inverted during patient movement, air may enter the fluid path.

Some infusion pumps are only detect air, but do not to remove it. When air is detected, therapy is interrupted and alarms are triggered. Intravenous infusion devices therefore incorporate an “air-in-line” alarm that detects gas as it passes through a narrow section of tubing. Because bubbles and air gaps are inevitable, these alarms may be frequent. They disrupt clinical workflows, burden nursing time, reduce patient comfort, and can be especially problematic during critical therapies. Accordingly, it would be of benefit to remove air from an IV line without disrupting the sterile fluid pathway.

In various embodiments, the system employs active pneumatic pressure control to ensure that a controlled pressure differential exists across the gas-selective membrane during operation. Passive air-elimination filters rely solely on incidental pressure conditions within the infusion system to drive gas removal. However, in many infusion scenarios, the pressure conditions within the fluid pathway are not constant and may vary depending on pump operation, flow rate, downstream resistance, patient vascular pressure, or transient flow interruptions. For example, the pressure downstream of an infusion pump may at times fall below atmospheric pressure, particularly when flow slows or stops, or when the patient line presents a lower pressure condition than the upstream components. Under such circumstances, a passive vent or hydrophobic membrane exposed to atmosphere may allow air to be drawn into the fluid pathway rather than expelled from it. As a result, passive systems cannot reliably guarantee that gas will always move out of the fluid path.

Various embodiments advantageously actively generate and regulate pneumatic pressure conditions to ensure that the fluid-side pressure exceeds the air-side pressure across the gas-selective membrane. By actively controlling the pressure differential, e.g., by applying vacuum to the air side or by maintaining positive pressure on the fluid side, the system ensures that gas transfer across the membrane consistently occurs in the intended direction. This active pneumatic control allows the system to maintain reliable air removal even as infusion conditions change, including variations in flow rate, container orientation, or downstream pressure conditions.

When air-in-line alarms do occur, a nurse may often disconnect the patient and manually remove the air, which can be challenging and may require excessive priming of the IV line. When the air has been sufficiently removed, the nurse must re-program the pump, sanitize all connections, and re-connect the patient. Some pumps even have a start-up time, where time must pass before the pump is infusing at the programmed rate. This all, of course, is heavily consuming on nursing resources and directly impactful to timely delivery of therapy to the patient.

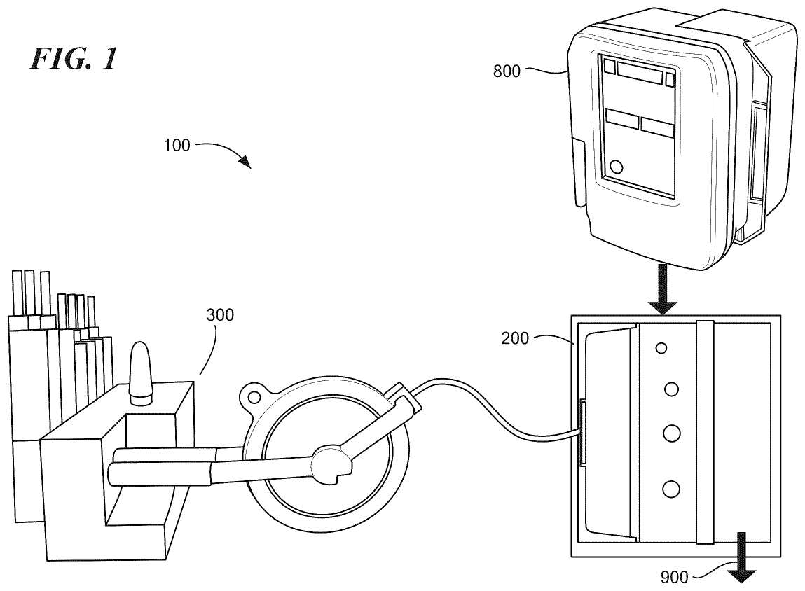

FIG. 1 schematically shows an example system 100 in accordance with various embodiments. The system 100 is configured to prevent hazardous air from entering a patient's vasculature, eliminate the need for air-in-line alarms and the associated workflow interruptions, prevent unintended cessation of fluid flow, and improve position independence and support greater patient mobility.

In illustrative embodiments, the system 100 includes an active sub-system of mechanical structures, controls, and sensors which may be referred to as Active Air Management (AAM). The AAM may be integrated as a subassembly within an infusion control system for removing air-in-line during applications such as infusion therapy. Additionally, or alternatively, the AAM may be configured as a standalone device for air removal.

For example, FIG. 1 shows the fluid delivery system 100 having an AAM subassembly 200, a pneumatic control sub-system 300, and an infusion controller 800, configured to deliver fluids to a patient 900.

Some embodiments include a modified air-eliminating filter that has a hermetically sealed chamber. Unlike filters that are open to atmosphere, the sealed chamber enables precise control of both positive pressure and vacuum.

Illustrative embodiments may include a pneumatic control system configured to precisely regulate pressure within the pneumatic housing of the Active Air Management (AAM) subassembly. In some embodiments, the pneumatic control system is configured to generate and maintain a controlled vacuum within the housing. In other embodiments, the system is configured to generate and maintain a controlled positive gas pressure. The pneumatic control system may include one or more pumps, regulators, pressure reservoirs, and electronically actuated valves that cooperate to achieve stable and responsive pressure control under varying operating conditions.

A valving system may be provided to selectively isolate the pneumatic housing from the surrounding environment and from other pneumatic components. This selective isolation allows the system to maintain a sealed, hermetically controlled chamber, thereby enabling accurate pressure regulation and measurement without interference from ambient atmospheric conditions.

A sensing system may be integrated into the pneumatic housing to continuously monitor pressure conditions. The sensing system may include one or more pressure transducers configured to detect absolute pressure, differential pressure, or pressure rate-of-change within the housing. These measurements may be sampled in real time and transmitted to a computational system.

The computational system may be configured to analyze measured pressure data to determine changes associated with air transfer across an integral gas-selective filter. For example, as gas passes from the fluid path through the gas-selective membrane and into the pneumatic housing, the resulting pressure variation may be detected and quantified. Based on known chamber volume, temperature, and gas laws, the computational system may calculate the quantity or rate of air entering the housing from the fluid path. This quantitative determination enables active monitoring of air removal performance.

In various embodiments, gas removal across the gas-selective membrane occurs when a pressure differential is established such that the pressure on the fluid side of the membrane exceeds the pressure on the air side of the membrane. When this condition exists, gas contained within the fluid pathway is driven across the membrane from the higher-pressure fluid region to the lower-pressure air region, while the liquid phase is retained by the gas-selective properties of the membrane. The pressure relationship may be produced in multiple ways.

In some embodiments, a vacuum is applied to the air side of the membrane so that the air-side pressure is reduced below the pressure of the fluid path. In other embodiments, positive pressure is applied to the fluid side of the membrane so that the fluid-side pressure is elevated above the pressure present on the air side, which may be atmospheric or otherwise controlled. Regardless of the specific implementation, the fluid-side pressure exceeds the air-side pressure across the membrane, thereby creating a driving force that causes gas bubbles contacting the membrane surface to pass through the membrane and exit the fluid pathway. By explicitly establishing this pressure relationship as the governing operating condition, the system encompasses embodiments that generate the pressure differential through vacuum, positive pressure, or combinations of both, while relying on the same underlying principle of controlled pneumatic pressure differential across a gas-selective membrane to remove air from a flowing fluid stream.

In various embodiments, a control system may be configured to re-establish or adjust the target pressure level when a predetermined threshold is reached. For example, if vacuum pressure is reduced beyond a defined limit due to accumulated gas in the housing, the control system may activate a pump or open a valve to restore the desired vacuum level. Similarly, in positive-pressure embodiments, the control system may increase or stabilize pressure to maintain effective operation. Thresholds may be predetermined based on safety parameters, performance targets, or operational efficiency criteria.

A communication system may interface the AAM subassembly with one or more external control devices, such as an infusion control system. Through wired or wireless communication pathways, the AAM may transmit operational status, measured pressures, calculated air volumes, alarm conditions, and system diagnostics. This integration enables coordinated system behavior, enhanced safety monitoring, and improved clinical workflow.

Accordingly, as schematically illustrated in FIG. 1, a controlled vacuum may be applied to the outer surface of a gas-selective filter. Gas that contacts the inner surface of the gas-selective membrane from within the fluid path may pass across the membrane and enter the vacuum chamber. The system may detect reductions in vacuum level caused by this gas transfer and quantify the corresponding air removal. In some embodiments, a gas-selective membrane may be in the form of a hydrophobic membrane, an oliophobic membrane, or membranes that otherwise allow the movement of gas without being wetted or blocked by the nature of the liquid. These membranes may be referred to as omniphobic and may be created with a combination of materials, geometries, and chemical treatments

The vacuum applied to the outer surface of the gas-selective filter may then be restored to its target level, allowing continued removal of gas from the fluid path. By cyclically or continuously restoring vacuum, the system enables removal of an effectively unlimited quantity of gas without interrupting fluid delivery.

In alternative embodiments, positive pressure may be applied in a manner that directs gas within the fluid path into contact with the inner surface of the gas-selective membrane, thereby promoting gas transfer across the membrane. In such embodiments, internal pressure conditions may be measured and adjusted as necessary to ensure sustained and controlled gas removal.

In various embodiments, the computational results generated by the AAM—including calculated air volume, rate of air removal, pressure trends, and system status—may be communicated to one or more infusion control devices or supervisory systems. This communication supports integrated monitoring, documentation, and automated control responses where appropriate.

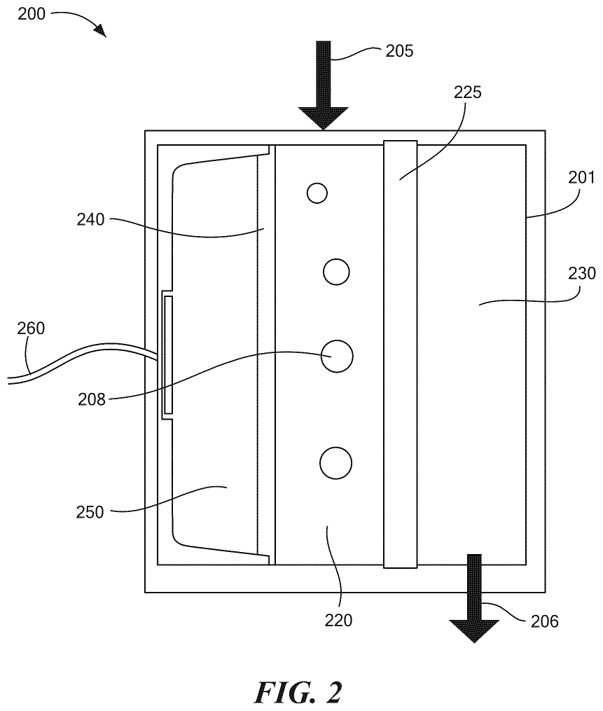

FIG. 2 schematically shows an example mechanical construction of a filter subassembly in accordance with various embodiments. In various embodiments, the filter subassembly is the AAM subassembly 200 as shown in FIG. 1.

As shown in FIG. 2, the Active Air Management (AAM) subassembly 200 includes a hermetically sealed housing 201 that defines multiple internal chambers configured to separate liquid and gas components of a fluid stream. The housing 201 may be formed from a rigid or semi-rigid biocompatible material and sealed to prevent unintended gas exchange with the external environment. By maintaining a sealed architecture, the AAM subassembly enables precise pneumatic control and accurate measurement of gas removal.

A fluid source 205, which may include an upstream IV bag, syringe, pump-driven infusion line, or other medical fluid reservoir, communicates with a fluid chamber 220 of the housing 201. The fluid chamber 220 receives a flowing mixture of the desired therapeutic liquid and entrained or introduced air bubbles 208. These air bubbles may originate from upstream line priming, fluid container changes, cavitation events, or other routine clinical handling conditions.

Within the fluid chamber 220, gravitational, buoyant, and flow-dynamic effects may promote partial separation of the liquid and gas phases. In various embodiments, the chamber geometry is configured to migrate air bubbles 208 toward a region adjacent to a gas-selective membrane 240, while the liquid phase preferentially contacts a hydrophilic membrane 225 (when present).

In various embodiments, the geometry of the fluid pathway is configured to ensure that entrained air bubbles are compelled to physically contact the gas-selective membrane. Hydrophobic or otherwise gas-selective membranes permit gas transfer only when the gas phase directly engages the membrane surface. Therefore, the fluid pathway may be intentionally shaped to promote such contact. For example, the fluid pathway adjacent to the membrane may include a narrowed passage, a restricted cross-sectional area, a tortuous flow path, or other geometrical constraints that cause air bubbles to deform, elongate, or compress as they move through the channel. Because gas bubbles in a liquid are governed largely by surface tension and are readily deformable, constraining the flow path causes bubbles to expand laterally or otherwise increase their surface contact with adjacent structures, including the membrane surface.

In some embodiments, the pathway dimensions are selected such that bubbles above a predetermined threshold volume, such as bubbles exceeding approximately 10-20 microliters, cannot pass through the channel without contacting the membrane. In this way, the geometry of the fluid path acts as a mechanical forcing mechanism that directs bubbles toward the membrane and ensures reliable gas transfer when a pressure differential is present across the membrane.

In embodiments that include a hydrophilic membrane 225, the membrane is positioned to permit passage of liquid while substantially preventing the passage of air. The hydrophilic membrane 225 may be formed of a microporous material with surface energy characteristics that promote wetting by aqueous solutions. As liquid in the fluid chamber 220 contacts the hydrophilic membrane 225, it passes through the membrane into an outlet chamber 230. From the outlet chamber 230, the degassed liquid exits the AAM subassembly through a fluid outlet conduit 206 and proceeds downstream toward the patient.

Air bubbles 208 within the fluid chamber 220 are directed toward and contact a gas-selective membrane 240. The gas-selective membrane 240 may include a single membrane element or a plurality of membrane sections arranged to increase available surface area for gas transfer. The membrane material may be microporous and gas-permeable but resistant to liquid passage due to its gas-selective surface properties and defined pore size. When air bubbles 208 contact the inner surface of the gas-selective membrane 240, the gas phase passes across the membrane into a pneumatic chamber 250, while the liquid phase is retained within the fluid chamber 220.

The pneumatic chamber 250 is fluidly isolated from the liquid path and communicates with a pneumatic conduit 260. The pneumatic conduit 260 connects the pneumatic chamber 250 to the pneumatic control sub-system 300 described with reference to FIG. 1. Through this conduit, vacuum or positive pressure may be selectively applied and regulated within the pneumatic chamber 250 to facilitate gas transfer across the gas-selective membrane 240 and to enable measurement of gas accumulation. Pressure changes within the pneumatic chamber 250 may be monitored to quantify the amount of air removed from the fluid stream.

In some embodiments, the AAM subassembly 200 may be configured without a hydrophilic membrane 225. In such embodiments, the fluid chamber 220 may rely solely on the gas-selective membrane 240 for gas removal. The liquid flow path may be arranged so that fluid bypasses the gas-selective membrane's gas-permeable regions while still allowing air bubbles to contact the membrane surface. In this configuration, phase separation is achieved through membrane selectivity, chamber geometry, and controlled pneumatic conditions, without the need for a dedicated hydrophilic liquid-transfer membrane. In some embodiments, the system is configured to remove gas from the fluid pathway without the use of a hydrophilic liquid-transfer membrane. Some embodiments have an air-elimination filter with two membranes: a hydrophilic membrane that allows liquid to pass while blocking air bubbles, and a hydrophobic membrane that allows the trapped air to vent to atmosphere. However, such embodiments require relatively large membrane areas to maintain acceptable liquid flow rates and are susceptible to clogging, fouling, and compatibility limitations with certain fluids, including lipid-containing solutions, viscous formulations, or blood products.

In contrast, various embodiments may advantageously omit the hydrophilic membrane entirely and rely instead on controlled pneumatic pressure differentials and fluid-path geometry to remove gas. By actively establishing a pressure condition in which the fluid-side pressure exceeds the air-side pressure across the gas-selective membrane, and by configuring the fluid pathway so that bubbles are compelled to contact the membrane surface, entrained gas may be selectively removed through the gas-selective membrane while the liquid phase remains within the primary flow path. Accordingly, the gas-selective membrane may be relatively small because it does not serve as the primary liquid flow interface.

By integrating selective membranes, controlled pneumatic communication, and a hermetically sealed architecture, the AAM subassembly 200 provides active, measurable removal of air from a flowing medical fluid stream while maintaining continuous liquid delivery downstream.

In various embodiments, removal of gas from the fluid pathway does not rely on buoyancy or gravitational separation of air bubbles from the liquid phase. Instead, gas removal is driven primarily by a controlled pneumatic pressure differential established across a gas-selective membrane. When the pressure on the fluid side of the membrane exceeds the pressure on the air side, gas contained within the fluid stream is driven across the membrane and removed from the system while the liquid phase remains within the flow path. Because the mechanism of gas removal is governed by pressure differential and membrane contact rather than buoyant rise of bubbles within a chamber, the system does not require that air bubbles migrate upward or collect at a particular location within the device.

As a result, the system is capable of operating effectively regardless of device orientation. In various embodiments, the fluid delivery and air removal system may operate while positioned in a vertical orientation, horizontal orientation, inverted orientation, or any intermediate orientation encountered during normal clinical handling. This capability is particularly beneficial for ambulatory or mobile infusion applications in which the device may be carried by a patient, attached to clothing, or placed in a transport bag. Movement of the device or inversion of the fluid container may alter the distribution of gas within upstream components, for example, by exposing previously trapped gas pockets within a fluid reservoir to the fluid pathway. However, when such gas enters the flow path, the system continues to remove it through the gas-selective membrane as described herein. Accordingly, the system maintains effective air elimination even during motion, transport, or orientation changes, allowing reliable removal of entrained gas under conditions commonly encountered in hospital, home infusion, and ambulatory care environments.

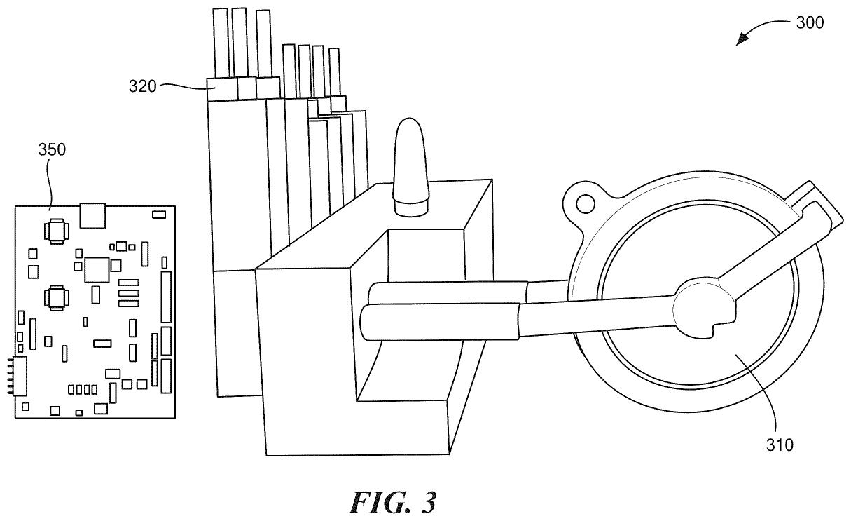

FIG. 3 schematically shows an example pneumatic control subsystem 300 in accordance with various embodiments.

In various embodiments, the pneumatic control sub-system 300 includes a microblower 310 configured to generate controlled pneumatic energy for operation of the AAM subassembly 200. The microblower 310 may be a piezoelectrically actuated device that vibrates at or near its mechanical resonant frequency (e.g., ultrasonic range) to produce high-frequency oscillating air pressure. These rapid oscillations can be fluidically rectified through internal flow-directing structures to generate a net directional airflow and corresponding positive pressure or vacuum. In certain embodiments, the microblower 310 is capable of generating pressures approaching approximately 1 bar, depending on system configuration and operating conditions.

The microblower 310 is dynamically coupled to the pneumatic work being performed. Under closed-loop control, when the pneumatic control sub-system 300 is regulating a specific target pressure (for example, maintaining a defined vacuum level or positive pressure within pneumatic chamber 250), the electrical power drawn by the microblower 310 is proportional to the actual pneumatic work required to move air. If airflow is minimal—such as when the flow path is blocked, sealed, or at equilibrium—the microblower draws minimal power because little or no air displacement is occurring. Conversely, if significant airflow is required, such as during active evacuation of accumulated gas from the pneumatic chamber, the electrical power consumption correspondingly increases. This tight coupling between power draw and fluidic workload provides inherent efficiency and diagnostic value.

The pneumatic control subsystem 300 may further include a pneumatic valve 320 that enables programmatic opening and closing of pneumatic flow paths between the microblower 310 and the pneumatic conduit 260 leading to the AAM subassembly. The valve 320 may be electronically actuated and may support multiple operational states, such as isolation, evacuation, pressurization, or venting. Through coordinated control of the microblower 310 and valve 320, the system may selectively apply vacuum, apply positive pressure, isolate the pneumatic chamber, or maintain static pressure conditions as required.

Electronic control circuitry and embedded firmware structures 350 may govern operation of the microblower 310 and pneumatic valve 320. These electronics may include a microcontroller, power management circuitry, driver electronics for piezoelectric actuation, pressure sensor interfaces, and analog-to-digital conversion components. The firmware may implement closed-loop control algorithms that continuously monitor pressure feedback from the AAM pneumatic chamber and dynamically adjust microblower drive amplitude, frequency, duty cycle, and valve state to maintain a desired pressure setpoint. Control strategies may include proportional-integral-derivative (PID) control, adaptive control routines, or threshold-based logic depending on system requirements.

The electronics 350 may also perform safety monitoring and diagnostics. For example, abnormal power consumption, unexpected pressure response, or failure to achieve a target pressure within a defined time window may trigger fault detection routines. These diagnostics can help identify conditions such as leaks, membrane saturation, blocked conduits, or component malfunctions.

Additionally, the electronics 350 provide communication capabilities to the infusion controller 800. Communication may occur through wired interfaces (such as serial communication, I2C, SPI, CAN, or other digital protocols) or through other defined communication architectures within Fluid Delivery System 100. Operational parameters transmitted to the infusion controller 800 may include real-time pressure measurements, calculated air removal volumes, system status, alarm conditions, microblower power consumption metrics, and diagnostic data. This integration allows coordinated system behavior, supports clinical decision-making, and enables logging or documentation of air removal activity during infusion therapy.

Through the coordinated interaction of the microblower 310, pneumatic valve 320, and control electronics 350, the pneumatic control sub-system 300 provides precise, efficient, and dynamically responsive pneumatic regulation to support active air management within Fluid Delivery System 100.

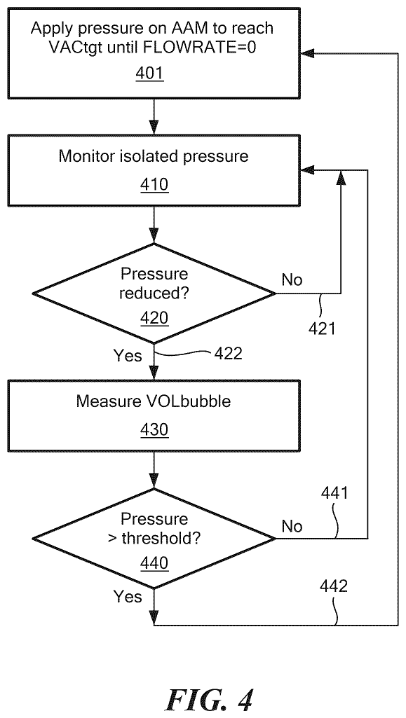

FIG. 4 shows an example process of automatic detection, measurement, and elimination of air from a fluid pathway in accordance with various embodiments. In various embodiments, the process is described in the context of Fluid Delivery System 100, including AAM subassembly 200 and pneumatic control sub-system 300.

It should be understood that the sequence of steps shown in FIG. 4 is exemplary. Additional steps may be included, certain steps may be omitted, and the order of execution may vary depending on system configuration or control strategy. Some steps may be performed concurrently, and others may be optional in particular embodiments. Accordingly, the process represents one illustrative implementation of active air management and may be modified by those skilled in the art without departing from the intended scope.

At step 401, the active air elimination process begins by commanding pneumatic control sub-system 300 to establish a target pressure within pneumatic chamber 250. The target pressure, denoted VACtgt, may be either a negative gauge pressure (vacuum) or a positive gauge pressure, depending on the embodiment. For example, in a vacuum-based implementation, VACtgt may be approximately −2.0 PSIg, although other values may be selected based on membrane characteristics, chamber volume, and desired air removal performance.

The power delivered to pneumatic control sub-system 300 (including microblower 310) may be regulated using a closed-loop control algorithm, such as a proportional-integral-derivative (PID) controller implemented in electronics and firmware 350. Pressure sensor feedback from pneumatic chamber 250 is continuously compared to VACtgt, and the drive amplitude, frequency, or duty cycle of microblower 310 is adjusted accordingly to minimize error.

During this step, electrical power consumption of the microblower may also be monitored. Because the microblower's power draw is proportional to pneumatic work, power levels decrease as airflow approaches zero. When the target pressure is reached and airflow across gas-selective membrane 240 ceases (i.e., a zero-flow condition), the system stabilizes at minimal steady-state power, indicating that the chamber has reached equilibrium.

In embodiments utilizing positive pressure rather than vacuum, the system applies a controlled positive gauge pressure to the source fluid upstream of the gas-selective membrane. Downstream flow resistance may be actively adjusted to achieve a desired liquid flow rate while maintaining sufficient pressure differential across the membrane to promote gas transfer.

A narrowed or controlled flow path geometry may be used so that air bubbles above a predetermined size (e.g., approximately 15 μL) are forced into mechanical contact with gas-selective membrane 240. The pressure differential across the membrane drives the gas phase from the relatively higher-pressure fluid side to the lower-pressure air side of the membrane (which may be atmospheric pressure or a controlled pneumatic region). Liquid is retained due to the gas-selective and microporous properties of the membrane.

Air passing through the gas-selective membrane may be vented to atmosphere or directed into a sequestration container. To prevent retrograde air flow from atmosphere into the fluid pathway, a one-way check valve may be incorporated on the air side of the membrane.

In such embodiments, the pressure of the source fluid is precisely monitored. A measurable increase in downstream liquid flow, or a transient pressure change, may indicate that entrained air has exited the fluid path. The system may increase downstream resistance to the point of temporarily halting liquid flow below the air elimination system, thereby allowing removal of accumulated air without advancing fluid toward the patient. This configuration enables removal of a substantial or effectively unlimited quantity of air while maintaining controlled delivery conditions.

Following stabilization of the target pressure at step 401, step 410 isolates pneumatic chamber 250 by closing valve 320. Once isolated, the chamber becomes a sealed measurement volume. Electronics 350 passively monitor the pressure signal within the chamber without actively driving the microblower.

This isolation allows small pressure changes resulting from gas transfer across gas-selective membrane 240 to be detected with high sensitivity.

At step 420, the monitored pressure is evaluated. If the pressure has not changed significantly relative to a predefined tolerance band, the process proceeds along step 421 and returns to step 410 for continued passive monitoring. The loop between steps 410 and 420 represents a steady-state monitoring phase during which the system awaits detection of air ingress into pneumatic chamber 250.

If, at step 420, the system determines that the pressure has changed by more than a predefined threshold—indicating that gas has passed through gas-selective membrane 240—the process proceeds along step 422 to step 430.

At step 430, the system measures or calculates bubble size (or cumulative air volume). This calculation may be based on the ideal gas law or other gas-state relationships, taking into account, for example, one or more of the following: measured pressure change, known chamber volume, temperature (measured or assumed), and/or baseline pressure prior to isolation.

From these parameters, electronics 350 may compute the quantity of air that entered pneumatic chamber 250. The system may log this value, transmit it to infusion controller 800, and/or use it to determine subsequent control actions.

At step 440, the system determines whether the pressure (or vacuum) within pneumatic chamber 250 has deviated beyond a defined threshold relative to VACtgt. For example, if vacuum has diminished to approximately 50% of VACtgt, this may indicate that sufficient gas has accumulated to require re-evacuation.

If the threshold has been exceeded, the process proceeds along step 442 back to step 401, where pneumatic control sub-system 300 is reactivated to restore the target pressure level. This may involve reapplying vacuum or positive pressure as appropriate.

If, however, the pressure deviation is minor and remains below the threshold (e.g., less than 50% of VACtgt), the process proceeds along step 441 back to step 410 for continued passive monitoring. In various embodiments, this approach conserves energy and minimizes unnecessary actuation while maintaining continuous air detection capability.

In various embodiments, the process described in FIG. 4 may be repeated continuously throughout the infusion session. The cycle of pressurization, isolation, monitoring, detection, measurement, and restoration may occur as many times as needed until the fluid source is depleted, therapy is completed, or the system is otherwise deactivated.

Accordingly, through this closed-loop, cyclic control strategy, the system enables, for example automatic detection of entrained air, quantitative measurement of air volume, active removal of air without interrupting therapy, and ongoing verification of pneumatic and membrane performance. In addition, the process described above supports enhanced patient safety, reduced reliance on air-in-line alarms, and continuous fluid delivery during infusion therapy.

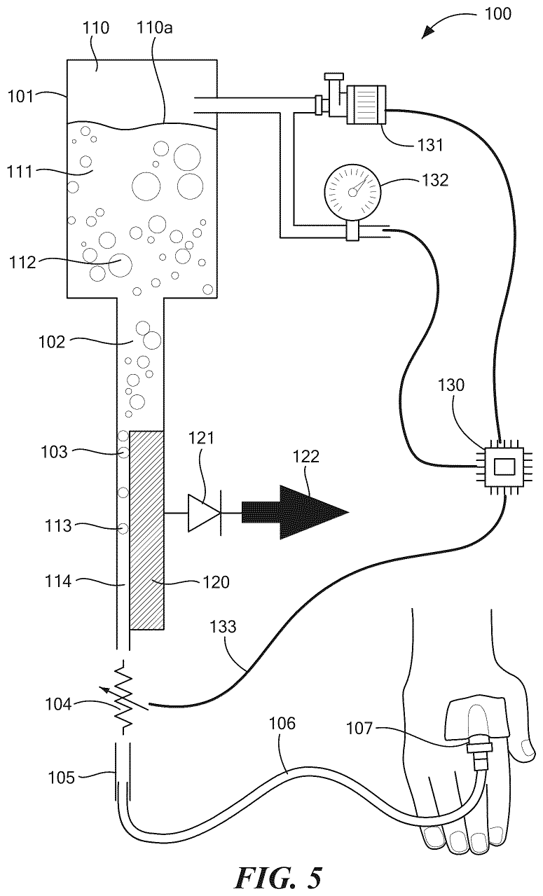

FIG. 5 shows another example system 100 in accordance with various embodiments. In various embodiment, the example system 100 shown in FIG. 5 may be a positive pressure system (e.g., positive pressure is applied to the fluid to cause air bubbles to engage with a gas-selective membrane).

FIG. 5 illustrates an embodiment of a fluid delivery and air elimination system configured to control fluid flow while actively separating and removing entrained air.

A fluid container 101, such as an IV bag, cartridge, or other therapeutic reservoir, supplies delivery fluid to the system. The fluid exits the container through a proximal fluid conduit 102, which directs the fluid toward a narrow fluid passage channel 103. The proximal conduit may be flexible medical-grade tubing and may include standard connectors, drip chambers, or upstream safety components depending on the clinical configuration.

The narrow fluid passage channel 103 is geometrically configured to constrain the cross-sectional area of the flowing stream. This reduced diameter promotes controlled fluid velocity and causes entrained air bubbles to elongate and increase in surface contact with adjacent membrane structures. By mechanically constraining bubble geometry, the system improves the likelihood that air bubbles will contact the gas-selective membrane surface for removal.

Downstream of the narrow passage channel 103, the fluid passes through a variable flow restrictor 104. The variable flow restrictor 104 may be an electronically actuated pinch valve, proportional valve, adjustable orifice, or similar controllable resistance element. It allows dynamic regulation of downstream flow resistance and therefore precise control of flow rate and upstream pressure conditions. After passing through the restrictor 104, the fluid enters a distal fluid conduit 105, continues into a patient line 106, and ultimately reaches the patient catheter 107 for infusion into the patient.

Surrounding or mechanically coupled to at least a portion of the delivery pathway is a pressurized fluid chamber including a pressurized fluid 110. Pressurized fluid 110—such as air or another gas—applies controlled external pressure to the delivery fluid region 111. This external pressure establishes a defined pressure differential across the system and may be used to drive flow, control bubble compression, and facilitate air removal through membrane structures. In some embodiments, a fluid separating membrane 110a may be positioned between the pressurized fluid 110 and the delivery fluid 111. This membrane permits pressure transmission while maintaining physical separation between the pressurizing medium and the therapeutic fluid.

In various embodiments, the system operates using a positive-pressure in which the pressure of the fluid upstream of the gas-selective membrane is actively maintained above the pressure present on the air side of the membrane. A pneumatic pressure source, such as a microblower, diaphragm pump, compressed gas source, or other pressure-generating mechanism, applies controlled positive pressure to the fluid pathway or to a chamber that mechanically transmits pressure to the fluid. This pressure may be applied directly to the fluid or indirectly through a flexible reservoir, diaphragm, or other pressure-transmitting structure. By maintaining the upstream fluid pressure above atmospheric pressure or above the pressure present on the air side of the membrane, a pressure differential is created across the gas-selective membrane. When air bubbles within the flowing fluid contact the membrane surface, the higher pressure on the fluid side drives the gas across the membrane while preventing liquid from passing through.

To maintain the desired pressure conditions during operation, the system may incorporate one or more downstream flow control elements that regulate resistance to fluid flow. For example, a variable flow restrictor, proportional valve, pinch valve, adjustable orifice, or other controllable restriction may be positioned downstream of the membrane region. By adjusting downstream resistance, the system can regulate flow rate while simultaneously maintaining a desired upstream pressure level that promotes effective gas removal. As bubbles are forced into contact with the gas-selective membrane by the constrained fluid path, the established pressure differential causes gas to pass through the membrane and exit the system. The removed gas may be vented to atmosphere through a one-way check valve or directed into a dedicated air capture volume, reservoir, or containment chamber. Through active control of upstream pressure and downstream flow resistance, the positive-pressure embodiment provides continuous, controlled gas removal while maintaining stable fluid delivery conditions within the infusion pathway.

Within the delivery fluid 111, source air bubbles 112 may be present. These bubbles can originate from upstream container changes, incomplete priming, dissolved gas coming out of solution, or handling of the fluid container. As the fluid enters the narrow fluid passage channel 103, the bubbles become geometrically constrained, forming restricted air bubbles 113. Constraining the bubbles increases their internal pressure and surface contact with adjacent membrane structures, improving the efficiency of air extraction.

Adjacent to the narrow passage channel 103 is a gas-selective membrane 120. The gas-selective membrane is gas-permeable but liquid-impermeable under normal operating pressures. When restricted air bubbles 113 contact the gas-selective membrane 120, the pressure differential across the membrane drives gas molecules through the membrane pores while retaining the liquid phase within the fluid pathway. As a result, a filtered liquid stream 114, substantially free of air bubbles, continues downstream toward the patient.

Air that passes through gas-selective membrane 120 is directed through a one-way air check valve 121. The check valve 121 ensures unidirectional flow of gas away from the fluid pathway and prevents retrograde air entry into the system. The removed air exits the system through an air exit port 122, which may vent to atmosphere or connect to a controlled air collection or sequestration chamber.

An electronic control system 130 coordinates pressure regulation and flow control. The electronic control system 130 may include a microcontroller, power management circuitry, and embedded control algorithms. It regulates the pressure of pressurized fluid 110 using an active fluid pressure pump 131, which may be a microblower, diaphragm pump, or other precision pneumatic actuator. Feedback from a pressure sensor 132 allows closed-loop control of the pressurized chamber, ensuring that target pressures are achieved and maintained.

In addition to pressure regulation, electronic control system 130 manages flow conditions by actuating the variable flow restrictor 104 through a restrictor control mechanism 133. In various embodiments, by adjusting downstream resistance, the system can control overall infusion rate, increase upstream pressure to promote bubble removal, temporarily slow or halt liquid flow to enhance air extraction efficiency, and/or maintain stable operating conditions during dynamic bubble removal events.

Through coordinated regulation of pressure and flow resistance, the system shown in FIG. 5 enables controlled delivery of therapeutic fluid while actively detecting, constraining, and removing entrained air before the fluid reaches patient catheter 107.

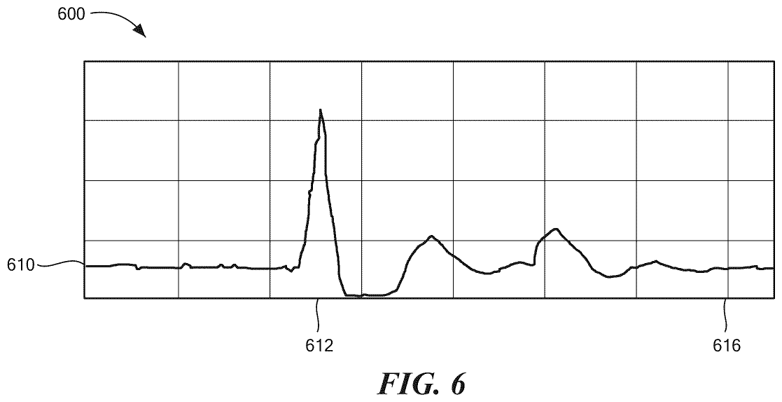

FIG. 6 schematically shows an example active gas monitoring, detecting, and measuring chart 600 in accordance with various embodiments.

As shown in FIG. 6, chart 600 illustrates the real-time power consumption of pneumatic control sub-system 300 over a short interval—on the order of approximately one second—during which one or more air bubbles transition from fluid chamber 220 into pneumatic chamber 250 through gas-selective membrane 240 (see FIG. 2). The chart demonstrates the dynamic relationship between pneumatic workload and electrical power draw of the microblower within the pneumatic control sub-system.

Under steady-state conditions, represented by power level 610, there is no net movement of air into pneumatic chamber 250. The target pressure has already been established, and no gas is crossing membrane 240. Because the microblower is tightly coupled to the actual pneumatic work being performed, the absence of airflow corresponds to minimal electrical power consumption. In this state, the blower maintains pressure equilibrium with only a low holding power requirement.

At time 612, a discrete air bubble crosses gas-selective membrane 240 and enters pneumatic chamber 250. This event creates a transient airflow through pneumatic conduit 260 as the system responds to the sudden influx of gas. The introduction of air temporarily alters the chamber pressure, causing the closed-loop control system to increase blower output in order to restore the target pressure level. As a result, the electrical power draw increases. The magnitude and duration of this power increase are proportional to the volume and rate of entry of the air bubble. For example, larger bubbles may result in higher and/or longer power spikes, while smaller bubbles may produce correspondingly smaller transient increases.

Between times 612 and 616, a series of smaller air bubbles enters pneumatic chamber 250. Each bubble crossing event produces a distinguishable power fluctuation in chart 600. These fluctuations appear as rapid, discrete peaks in power consumption, reflecting the real-time pneumatic work required to evacuate each increment of gas and re-establish the target pressure. Because the system continuously monitors both pressure and power, these transient power signatures may be correlated with calculated air volumes using known chamber parameters and gas relationships.

At time 616, all air bubbles have been removed from fluid chamber 220, and gas transfer across membrane 240 ceases. Airflow through conduit 260 stops, and the pressure within pneumatic chamber 250 stabilizes at its commanded setpoint. Consequently, the blower's power consumption returns to a low and steady baseline level, indicating that no additional pneumatic work is being performed.

Chart 600 therefore illustrates several operational characteristics of pneumatic control sub-system 300: power consumption is directly proportional to pneumatic work (i.e., air movement), discrete bubble events produce measurable and time-resolved power signatures, the system is capable of distinguishing between steady-state holding conditions and active air evacuation, and bubble size and frequency may be inferred from the amplitude and density of power transients.

This behavior provides both functional and diagnostic advantages. Functionally, it confirms that air removal is occurring, improving energy efficiency. Diagnostically, real-time power monitoring offers an additional modality for detecting membrane activity, identifying abnormal flow conditions, and validating proper operation of the AAM system.

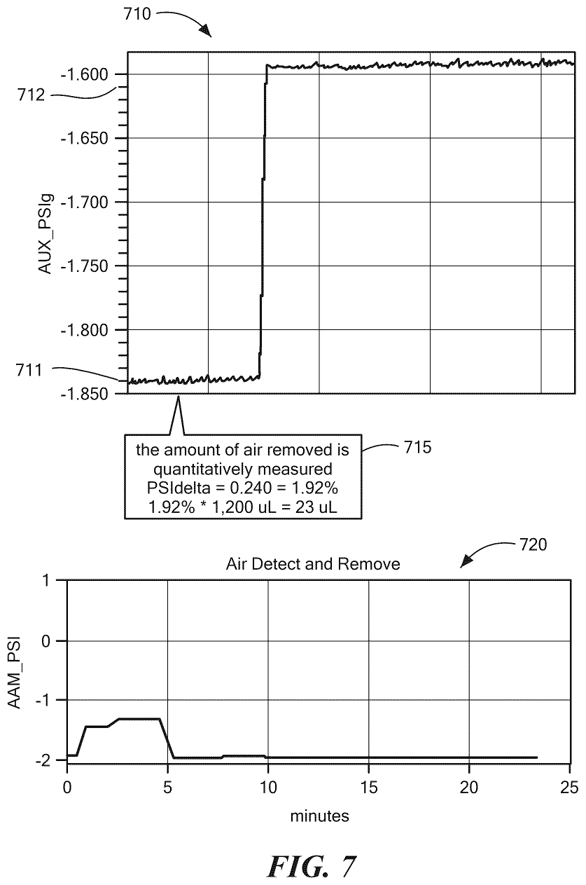

FIG. 7 schematically illustrates passive gas monitoring, detection, and quantitative measurement in accordance with illustrative embodiments. In this context, “passive” may refer to a measurement phase during which pneumatic chamber 250 is temporarily isolated (e.g., by closing valve 320), and no active pumping is occurring. The chamber therefore functions as a sealed reference volume, allowing small pressure changes caused by gas ingress to be detected with high sensitivity.

For example, pressure monitoring chart 710 represents high-speed pressure measurements obtained from a pressure sensor integrated into electronics 350 (see FIG. 3). The sensor measures pressure within conduit 260 and, by extension, within pneumatic chamber 250.

In the illustrated example, the chamber has previously been evacuated to a controlled vacuum level 711 of approximately −1.84 PSIg. This value represents a stable, isolated baseline condition in which no gas is actively crossing gas-selective membrane 240.

When an air bubble 208 passes from fluid chamber 220 into pneumatic chamber 250 through gas-selective membrane 240 (see FIG. 2), the newly introduced gas increases the absolute pressure inside chamber 250. Because the chamber is sealed during the passive monitoring interval, even a small volume of air produces a measurable pressure rise.

As shown at 712, the pressure increases from approximately −1.84 PSIg to approximately −1.61 PSIg within milliseconds. This transient pressure shift corresponds directly to the volume of the bubble that entered the chamber. The speed of this pressure change reflects both the responsiveness of the pressure sensor and the small volume of pneumatic chamber 250, which amplifies measurable pressure deviations.

The volume of the entering air bubble may be computed (as indicated at 715) using the Ideal Gas Law relationship. Because pneumatic chamber 250 has a known reference volume (for example, approximately 1,200 μL), and the temperature is either measured or assumed to be constant over the short monitoring interval, the change in pressure (ΔP) can be directly related to the incremental gas quantity or volume (ΔV) introduced into the chamber.

Using the measured pressure shift from −1.84 PSIg to −1.61 PSIg and the known chamber volume, the system may calculate that the single air bubble corresponds to approximately 23 μL at delivery pressure. Because the pressure measurement reflects actual in-system conditions, the computed bubble size represents its effective volume under infusion pressure rather than at atmospheric conditions. If desired, the system may normalize this value to standard temperature and pressure (STP) for logging or reporting consistency.

This quantitative capability enables the system not only to detect air presence, but also to measure individual bubble size and cumulative air volume with high resolution.

Chart 720 illustrates pressure monitoring over a longer timescale, potentially spanning minutes or hours of infusion. This longer-term trend provides insight into cumulative air removal behavior and overall system performance.

Between times 721 and 722, a series of air bubbles enters pneumatic chamber 250. Each bubble contributes incremental pressure increases, resulting in a gradual reduction of vacuum level over time. Rather than sharp isolated spikes, this longer view may show a stepped or sloped pressure profile reflecting multiple bubble events.

At time 722, the vacuum level has degraded to a predefined threshold (for example, 50% of the target vacuum). At this point, pneumatic control sub-system 300 reactivates and restores the vacuum to its commanded setpoint. This restoration appears as a rapid downward shift in pressure on the chart, returning the chamber to its baseline condition and preparing it for further passive monitoring.

Although FIG. 7 illustrates vacuum-based detection, the same measurement principles apply to embodiments utilizing positive pressure. For example, in a positive-pressure configuration, pneumatic chamber 250 may be pressurized to a target positive gauge pressure (e.g., +1.5 PSIg). Valve 320 isolates the chamber.

If gas passes into or out of the chamber across gas-selective membrane 240, the chamber pressure will change measurably. For example, if gas enters the chamber from the fluid path, the absolute pressure will increase further, while if gas leaves the chamber (depending on configuration), pressure may decrease.

As in the vacuum case, the magnitude of pressure change (ΔP) within the known reference volume can be used to calculate the corresponding gas quantity or volume using the Ideal Gas Law. The only difference is the direction of pressure deviation relative to baseline.

In positive-pressure systems, the pressure sensor measures increases above atmospheric pressure rather than reductions below atmospheric pressure. However, the mathematical relationship between pressure change and gas quantity or volume remains the same. Electronics 350 may include calibration routines to compensate for one or more of the following: sensor offset and drift, temperature variation, barometric pressure changes, and/or non-ideal gas behavior at higher pressures (if applicable).

Passive pressure monitoring as shown in FIG. 7 may provide high sensitivity to small bubble volumes due to sealed reference volume, quantitative measurement of individual bubble size, ability to log cumulative air removal over time, reduced energy consumption, since active pumping is paused during monitoring, and/or independent verification of membrane performance.

In various embodiments, by leveraging precise pressure sensing within a controlled pneumatic reference volume, the system may provide reliable detection and measurement of entrained air under both vacuum-based and positive-pressure operating modes.

In various embodiments, the system is configured not only to remove gas from the fluid pathway but also to detect and quantitatively measure air removal events. In certain implementations, this measurement capability is achieved through pressure-based monitoring within a sealed pneumatic chamber having a known reference volume. When the pneumatic chamber is isolated from the surrounding environment, for example by closing the valve, the chamber forms a sealed measurement volume. If gas passes through the gas-selective membrane and enters the chamber, the additional gas increases the pressure within the chamber. Because the chamber volume is known, even small quantities of gas produce measurable pressure changes. By monitoring these pressure variations with a pressure sensor, the system can detect the presence of gas entering the chamber and determine the quantity of gas removed from the fluid pathway.

In various embodiments, the volume of gas entering the chamber may be calculated using gas-state relationships such as the Ideal Gas Law or other thermodynamic relationships. Parameters used in these calculations may include the measured change in pressure within the chamber, the known chamber reference volume, the baseline pressure prior to gas entry, and the temperature of the gas within the chamber (which may be measured or assumed over short time intervals). Using these parameters, the system may calculate the incremental gas volume associated with an individual bubble event or determine the cumulative gas volume removed over time. Because the pressure measurement occurs within a controlled pneumatic reference volume, the resulting calculations can provide high sensitivity to relatively small bubble volumes.

In addition to pressure-based measurement, certain embodiments may employ power-based sensing to detect gas removal events. In these embodiments, the pneumatic actuator, such as a microblower, diaphragm pump, or other pressure-generating device, draws electrical power proportional to the pneumatic work required to move air. When gas enters the pneumatic chamber or flows through the pneumatic pathway, the actuator performs additional fluidic work, resulting in a transient increase in power consumption. Conversely, when airflow ceases and the pressure setpoint is maintained without additional air movement, the actuator consumes minimal power. By monitoring the electrical power consumption of the pneumatic actuator, the system can detect airflow events associated with gas removal.

These pressure and power signals may be monitored in real time. Transient pressure deviations within the sealed chamber may correspond to discrete gas-entry events, while transient power spikes may correspond to the pneumatic work required to evacuate gas or restore the chamber pressure. By analyzing these signals, the system may detect individual bubble events, calculate the approximate volume of each bubble, and determine the cumulative quantity of gas removed from the system over a defined time interval. This measurement capability enables several operational functions, including confirmation that air is being removed from the fluid pathway, detection of abnormal gas generation or outgassing within the fluid system, and verification that the air management components, including the gas-selective membrane and pneumatic control subsystem, are functioning properly during operation.

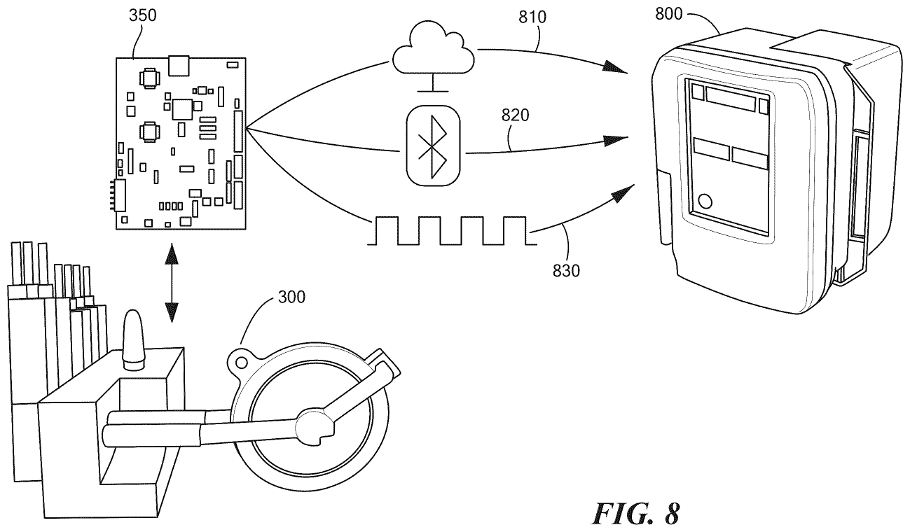

FIG. 8 schematically shows example details of a controller of FIG. 1 configured in accordance with illustrative embodiments of the invention. The controller 800 is shown as a collection of functional components operatively connected through a generalized interconnect mechanism. In the simplified representation of FIG. 8, this interconnect is depicted as a bus that enables communication among internal components. However, those skilled in the art will recognize that any suitable communication architecture may be used, including direct point-to-point connections, shared memory structures, switched interconnects, serial communication links, or distributed network topologies. The depiction of a bus is therefore illustrative only and not limiting.

It should also be understood that FIG. 8 is a high-level functional diagram. Each illustrated component may be implemented in a variety of conventional ways using hardware, software, firmware, or combinations thereof. For example, controller 800 may include one or more microprocessors or microcontrollers executing firmware instructions stored in non-volatile memory. In other embodiments, certain functions may be implemented using application-specific integrated circuits (ASICs), field-programmable gate arrays (FPGAs), discrete analog or digital components, or mixed-signal integrated circuits. Hybrid implementations are also contemplated, in which time-critical control loops are executed in dedicated hardware while supervisory logic and communications are handled in software.

Accordingly, the depiction of infusion controller 800 as a single block in FIG. 8 is for clarity and simplicity. In some embodiments, controller functionality may be physically distributed across multiple circuit boards, modules, or housings. For example, certain pneumatic control functions may reside within electronics 350 of pneumatic control sub-system 300, while higher-level infusion management, user interface control, and data logging functions may reside within a separate infusion pump controller enclosure. These components may communicate over wired or wireless links while collectively functioning as a unified control system.

It should be reiterated that FIG. 8 provides a simplified conceptual representation. A practical implementation of controller 800 may also include additional elements such as central processing units (CPUs), memory subsystems (including RAM and non-volatile storage), clock and timing circuits, watchdog timers, safety interlocks, analog front-end circuitry for sensor acquisition, motor or actuator drivers, display interfaces, user input interfaces, and power management circuitry. The figure is not intended to enumerate all such components but rather to highlight functional relationships relevant to the Active Air Management (AAM) system.

In various embodiments, pneumatic control sub-system 300 communicates with electronics 350 to monitor and analyze the performance of Active Air Management subassembly 200. Electronics 350 process pressure measurements, power consumption data, bubble detection events, calculated air volumes, and system diagnostics. This information may be periodically or continuously communicated to infusion controller 800.

Communication between electronics 350 and infusion controller 800 may occur through a variety of mechanisms, including: Wi-Fi communication (810) to a cloud-based computing platform or hospital network infrastructure; Bluetooth communication (820) for local wireless data exchange; Wired serial communication (830) such as USB, I2C, SPI, UART, CAN, or other suitable digital interfaces.

In some embodiments, communication protocols may include encryption, authentication, error detection, and redundancy to support regulatory and cybersecurity requirements applicable to medical devices.

The information transmitted from electronics 350 to infusion controller 800 may serve multiple clinical, operational, and safety purposes. For example, it may be used to perform one or more of the following: verify proper operation of the air-in-line detection and removal system; provide an audit log documenting all detected and removed air volumes during an infusion session; supply correlative data for comparison with real-time flow sensing measurements within the infusion control device; confirm completion of a programmed infusion volume by reconciling delivered fluid volume with measured air removal; confirm that the hydrophilic membrane element (when present) within the AAM has been fully wetted and is functioning properly; detect abnormal conditions such as membrane degradation, leakage, blockage, or unexpected air ingress; and/or support preventative maintenance, calibration tracking, and system diagnostics.

In some embodiments, infusion controller 800 may use AAM data in closed-loop coordination with its own flow control algorithms. For example, detection of unusually large or frequent air bubbles may prompt temporary adjustments in flow rate, alarm generation, or clinician notification.

Through this integrated architecture, controller 800 not only manages infusion parameters but also supervises and validates the performance of the AAM system, thereby enhancing patient safety, improving documentation, and supporting reliable long-term operation.

In certain implementations, a user interface can include one or more physical interface devices such as input devices, output devices, and combination input/output devices and a software stack configured to drive operation of the devices. These user interface elements may render visual, audio, and/or tactile content. Thus the user interface may receive input or provide output, thereby enabling a user to interact with the controller.

In some embodiments, the controller can also include at least one battery configured to provide power to one or more components integrated in the controller. The battery can include a rechargeable multi-cell battery pack. In one example implementation, the battery can include three or more lithium ion cells that provide electrical power to the other device components within the controller. In certain implementations, the battery capacity, runtime, and type (e.g., lithium ion, nickel-cadmium, or nickel-metal hydride) can be changed to best fit the specific application of the controller.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, programmable analog circuitry, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

In some implementations, the controller includes one or more processors (or one or more processor cores) that each are configured to perform a series of instructions that result in manipulated data and/or control the operation of the other components of the controller. In some implementations, when executing a specific process processor can be configured to make specific logic-based determinations based on input data received, and be further configured to provide one or more outputs that can be used to control or otherwise inform subsequent processing to be carried out by the processor and/or other processors or circuitry with which processor is communicatively coupled. Thus, the processor reacts to specific input stimulus in a specific way and generates a corresponding output based on that input stimulus. In some example cases, the processor can proceed through a sequence of logical transitions in which various internal register states and/or other bit cell states internal or external to the processor may be set to logic high or logic low. As referred to herein, the processor can be configured to execute a function where software is stored in a data store coupled to the processor, the software being configured to cause the processor to proceed through a sequence of various logic decisions that result in the function being executed. The various components that are described herein as being executable by the processor can be implemented in various forms of specialized hardware, software, or a combination thereof. For example, the processor can be a digital signal processor (DSP) such as a 24-bit DSP processor. The processor can be a multi-core processor, e.g., having two or more processing cores. The processor can be an Advanced RISC Machine (ARM) processor such as a 32-bit ARM processor. The processor can execute an embedded operating system, and include services provided by the operating system that can be used for file system manipulation, display & audio generation, basic networking, firewalling, data encryption and communications.

Illustrative embodiments of the invention are implemented as a computer program product having a computer usable medium with computer readable program code thereon. The computer readable code may be read and utilized by a computer system in accordance with conventional processes.

As used in this specification and the claims, the singular forms “a,” “an,” and “the” refer to plural referents unless the context clearly dictates otherwise. For example, reference to “the microblower” in the singular includes a plurality of microblowers, and reference to “the filter” in the singular includes one or more filters and equivalents known to those skilled in the art. Thus, in various embodiments, any reference to the singular includes a plurality, and any reference to more than one component can include the singular.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein.

It is, therefore, to be understood that the foregoing embodiments are presented by way of example only and that, within the scope of the appended claims and equivalents thereto, inventive embodiments may be practiced otherwise than as specifically described and claimed. Illustrative embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure. Disclosed embodiments, or portions thereof, may be combined in ways not listed above and/or not explicitly claimed. Thus, one or more features from variously disclosed examples and embodiments may be combined in various ways. For example, it is to be understood that the present disclosure contemplates that, to the extent possible, one or more features of any embodiment can be combined with one or more features of any other embodiment.

Various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Although the above discussion discloses various exemplary embodiments of the invention, it should be apparent that those skilled in the art can make various modifications that will achieve some of the advantages of the invention without departing from the true scope of the invention.

What is claimed is:

1. A system for detecting, measuring, and removing gas from a medical fluid pathway, the system comprising:
a fluid delivery path configured to convey therapeutic liquid from a fluid source toward a patient;
an Active Air Management (AAM) subassembly comprising:
a fluid chamber in fluid communication with the fluid delivery path and configured to receive the therapeutic liquid and any entrained gas; and
a pneumatic chamber separated from the fluid chamber by an integral gas-selective membrane that is configured to allow gas to pass from the fluid chamber into the pneumatic chamber while retaining liquid in the fluid chamber;
a pressure controller fluidly coupled to the pneumatic chamber and configurable to impose and maintain a controlled negative pressure differential between the pneumatic chamber and the fluid chamber;
a valve configured to selectively isolate the pneumatic chamber from the pressure controller to create a sealed measurement volume;
a pressure sensor configured to measure pressure changes within the sealed measurement volume chamber; and
a controller configured to calculate a gas quantity corresponding to the measured pressure change, and in response to the pressure change meeting or exceeding a threshold, cause the pressure controller to re-establish or adjust the controlled pressure differential in the pneumatic chamber.

2. The system of claim 1, wherein the pressure controller is configured to impose a negative gauge pressure within the pneumatic chamber relative to the fluid chamber to draw gas across the gas-selective membrane.

3. The system of claim 2, wherein the controller is configured to isolate the pneumatic chamber using the valve while the pneumatic chamber is at the negative gauge pressure, and to calculate the gas quantity based on a rise in pressure within the sealed measurement volume.

4. The system of claim 2, wherein the controller is configured to control the pressure controller to re-establish the negative gauge pressure when the measured pressure within the pneumatic chamber deviates from a target pressure by more than a predetermined percentage.

5. The system of claim 1, wherein the pressure controller is configured to impose a positive gauge pressure within the pneumatic chamber relative to atmospheric pressure.

6. The system of claim 5, wherein the positive gauge pressure establishes a pressure differential across the gas-selective membrane that promotes transfer of gas from the fluid chamber into the pneumatic chamber while substantially preventing passage of liquid.

7. The system of claim 5, wherein the controller is configured to calculate the gas quantity based on a change in the positive gauge pressure within the pneumatic chamber when the pneumatic chamber is isolated by the valve.

8. The system of claim 1, wherein the pressure controller comprises a piezoelectrically actuated microblower configured to generate oscillating air pressure that is rectified to produce the controlled pressure differential.

9. The system of claim 1, wherein the controller is configured to calculate the gas quantity using a known reference volume of the pneumatic chamber and a change in measured pressure in accordance with a gas law relationship.

10. The system of claim 1, further comprising a communication interface configured to transmit at least one of calculated gas quantity, pressure data, or system status information to an infusion control device.

11. A system for detecting, measuring, and removing gas from a medical fluid pathway, the system comprising:
a fluid delivery path configured to convey therapeutic liquid from a fluid source toward a patient;
a fluid chamber forming at least a portion of the fluid delivery path and configured to receive therapeutic liquid and entrained gas;
a gas-selective membrane positioned adjacent to the fluid chamber and configured to permit gas to pass therethrough;
a pressurized chamber containing a pressurizing medium and positioned to apply positive pressure to at least a portion of the fluid delivery path and a controllable fluid restrictor, so as to establish a pressure differential across the gas-selective membrane that promotes transfer of gas from the fluid chamber through the gas-selective membrane;
a pressure controller configured to regulate the positive pressure applied by the pressurized chamber;
and a fluid flow resistance control device to create a hydrodynamic backpressure, configured to regulate the positive pressure applied by the pressurized chamber;
a pressure sensor configured to measure pressure associated with at least one of the pressurized chamber or the fluid chamber; and
a controller configured to calculate a gas quantity based on a change in measured pressure.

12. The system of claim 11, wherein the pressurized chamber surrounds at least a portion of the fluid chamber and transmits pressure to the therapeutic liquid through a compliant wall or a separating membrane.

13. The system of claim 11, wherein the gas-selective membrane is positioned adjacent to a narrowed fluid passage configured to mechanically constrain air bubbles and increase contact between the air bubbles and the gas-selective membrane.

14. The system of claim 11, further comprising a variable flow restrictor positioned downstream of the fluid chamber and configured to selectively adjust flow resistance in the fluid delivery path.

15. The system of claim 14, wherein the controller is configured to increase downstream flow resistance to temporarily reduce liquid flow while gas is being transferred across the gas-selective membrane.

16. The system of claim 11, further comprising a one-way check valve positioned downstream of the gas-selective membrane and configured to prevent retrograde gas flow into the fluid chamber.

17. The system of claim 11, wherein the pressure controller comprises a pump configured to generate positive gauge pressure within the pressurized chamber.

18. The system of claim 11, wherein the controller is configured to calculate the gas quantity using a known reference volume and a pressure change in accordance with a gas law relationship.

19. The system of claim 11, wherein the controller is configured to communicate calculated gas quantity data to an infusion control device.

20. A method for detecting, measuring, and removing gas from a medical fluid pathway, the method comprising:
conveying a therapeutic liquid from a fluid source toward a patient through a fluid delivery path;
directing the therapeutic liquid and any entrained gas into a fluid chamber in fluid communication with the fluid delivery path;
drawing gas from the fluid chamber into a pneumatic chamber through an integral gas-selective membrane that allows gas to pass while retaining liquid in the fluid chamber by imposing and maintaining a controlled negative pressure differential between the pneumatic chamber and the fluid chamber using a pressure controller fluidly coupled to the pneumatic chamber;
selectively isolating the pneumatic chamber from the pressure controller using a valve to create a sealed measurement volume;
measuring pressure changes within the sealed measurement volume using a pressure sensor; and
calculating, with a controller, a gas quantity corresponding to the measured pressure change and, in response to the pressure change meeting or exceeding a threshold, causing the pressure controller to re-establish or adjust the controlled pressure differential in the pneumatic chamber.
